(19) United States
(12) Patent Application Publication
(10) Pub. No.: US 2003/0054344 A1
(43) Pub. Date: Mar. 20, 2003

(54) Method for generating ultra-fine spotted arrays

(76) Inventor: Francis M. Rossi, Menlo Park, CA (US)

Correspondence Address: Jeffrey B. Oster, Combimatrix Corporation, 6500 Harbour Heights Parkway, Mukilteo, WA, 98275, US

(21) Appl. No.: 09/522,554

(22) Filed: Mar. 10, 2000

Related U.S. Application Data
(60) Provisional application No. 60/123,992, filed on Mar. 11, 1999.

(51) Int. Cl. C12Q 1/68; G01N 33/53; G01N 33/542; G01N 33/543; B05D 3/00

(52) U.S. Cl. 435/6; 435/7.9; 427/2.11

(57) ABSTRACT

The present invention features a method for producing fine spotted arrays of molecules comprising the following steps of (a) immobilizing affinity anchors onto the array; (b) preparing molecules of interest having affinity for the anchors; (c) contacting the molecules of interest with the array; and (d) washing the array to remove unbound molecules of interest.

The present application claims priority to U.S. Provisional Application Serial No. 60/123,992 filed Mar. 11, 1999.

### FIELD OF THE INVENTION

The present invention is in the field of biological and chemical synthesis and processing. The present invention relates to methods for generating ultra-fine spotted microarrays featuring a greater density of molecules than previously possible.

### BACKGROUND OF THE INVENTION

Advances are continually emerging in the field of biological and chemical processing and synthesis equipment. Many novel and improved arrays or “gene chips” are being developed providing rapid methods for synthesizing chemical and biological materials. Examples of such technologies include those described by Pirrung et al., U.S. Pat. No. 5,143,854, those described by Southern in WO 93/22480, those described by Heller in WO 95/12808, those described in U.S. Pat. No. 5,849,486, those described in U.S. Pat. No. 5,632,957, those described in U.S. Pat. No. 5,605,662 and those described by Montgomery in WO 98/01221. The disclosure of the foregoing are herein incorporated by reference in their entirety. Methods for synthesizing chemical and biological materials may employ, for example, photolithographic techniques or electrochemical techniques.

Methods of preparing large numbers of different ligands have been painstakingly slow and prohibitively expensive when used at a scale sufficient to permit effective rational or random screening. For example, the method described by Merrifield et al, J. Am. Chem. Soc. 85:2149-2154 (1963) has been used to synthesize peptides on solid supports. In this method, an amino acid is bound covalently to a support made of an insoluble polymer. Another amino acid with an alpha protected group is reacted with the covalently bonded amino acid to form a dipeptide. After washing, the protective group is removed and a third amino acid with an alpha protective group is added to the dipeptide. This process is continued until a peptide of a desired length and sequence is obtained. Using the Merrifield method, synthesis of more than a handful of peptide sequences in a day is not technically feasible or economically practical.

To synthesize larger numbers of polymer sequences, it has been proposed to use a series of reaction vessels for polymer synthesis. For example, a tubular reactor system may be used to synthesize a linear polymer on a solid phase support by automated sequential addition of reagents. This method, however, also does not enable the synthesis of a sufficiently large number of polymer sequences for effective and economical screening.

Another method of preparing a plurality of polymer sequences uses a porous container enclosing a known quantity of reactive particles, larger in size than pores of the container. The particles in the containers may be selectively reacted with desired materials to synthesize desired sequences of product molecules. However, as with the other methods known in the art, this method is not practical for the synthesis of a sufficient variety of polypeptides for effective screening.

Other techniques have also been described and attempted. Several of these methods include synthesis of peptides on 96 plastic pins that fit the format of standard microtiter plates. Unfortunately, while these techniques have been somewhat useful, substantial problems remain. Methods using standard microtiter plates continue to be limited in the diversity of sequences that can be synthesized and screened. Although it is recognized that using microtiter plates produces essentially pure polymers because each polymer is synthesized in an isolated well of the microtiter plate, the number of polymers that can be produced in any given time is limited by the number of wells in a microtiter plate, i.e., 96. Moreover, the equipment needed for synthesis in the microtiter plates is large. Because of this limitation, use of microtiter plates requires a large amount of space to produce a relatively small number of peptides.

One attempt at synthesizing a large number of diverse arrays of polypeptides and polymers in a smaller space is found in U.S. Pat. No. 5,143,854 granted to Pirrnung et al. (1992). This patent describes the use of photolithographic techniques for the solid phase synthesis of arrays of polypeptides and polymers. The disclosed technique uses “photomasks” and photolabile protecting groups for protecting the underlying functional group. Each step of the process requires the use of a different photomask to control which regions are exposed to light and thus deprotected. The necessity of having to fabricate a new set of photomasks for each array of chemical monomers results in a method that is extremely expensive and not well-suited to automation. Moreover, this method is tedious and time consuming because each step of the synthesis requires the mechanical removal, replacement and realignment of a photomask. Thus, synthesizing a large number of libraries of polymers with the Pirrung method is an inefficient and uneconomical process.

Another drawback of the Pirrung method is that the photolabile protecting groups used cannot be removed as effectively as conventional acid or base labile protecting groups can be removed and are plagued by contamination due to undesired side reactions. Consequently, using Pirrung's method, the purity of the chemical array is often compromised due to incomplete removal of the protecting groups and subsequent failure of the underlying functional groups to react with the desired monomer, as well as contamination from undesired side reactions.

Another attempt to synthesize large numbers of polymers is disclosed by Southern in International patent application WO 93/22480, published Nov. 11, 1993. Southern describes a method for synthesizing polymers at selected sites by electrochemically modifying a surface; this method involves providing an electrolyte overlaying the surface and an array of electrodes adjacent to the surface. In each step of Southern's synthesis process, an array of electrodes is mechanically placed adjacent the points of synthesis, and a voltage is applied that is sufficient to produce electrochemical reagents at the electrode. The electrochemical reagents are deposited on the surface themselves or are allowed to react with another species, found either in the electrolyte or on the surface, in order to deposit or to modify a substance at the desired points of synthesis. The array of electrodes is then mechanically removed and the surface is subsequently contacted with selected monomers. For subsequent reactions, the array of electrodes is again mechanically placed adjacent the surface and a subsequent set of selected electrodes activated.

This method requires that a large amount of control be exercised over the distance that exists between the electrode array and the surface where synthesis occurs. Control over the distance between the electrodes and the surface for modification is required to ensure precise alignment between the electrodes and the points of synthesis and to limit the extent of diffusion of electrochemically generated reagents away from the desired points of synthesis. However, the inherent difficulty in positioning electrodes repeatedly and accurately within a few microns of the surface frequently results in the production of electrochemically generated reagents at undesirable synthesis points. Moreover, the diffusion of the electrochemically generated reagents from desired sites of reaction to undesired sites of reaction results in “chemical cross-talk” between synthesis sites. This cross-talk severely compromises the purity of the final product, as undesired binding reactions occur at unselected sites. The amount of cross-talk is further aggravated by the disruptions of surface tension that occur whenever the electrodes are moved, leading to convective mixing that causes increased movement of the electrochemically generated reagents. While Southern attempts to minimize the amount of cross-talk by applying a potential designed to counteract diffusion, as a practical matter, the electric fields Southern can generate are too low to prevent diffusion. When the potential is raised to increase the electric field, large quantities of undesired electrochemically generated reagents are produced. Hence, Southern is not a practical method for generating large numbers of pure polymers.

A more recent attempt to automate the synthesis of polymers is disclosed by Heller in International patent application WO 95/12808, published May 11, 1995. Heller describes a self-addressable, self-assembling microelectronic system that can carry out controlled multi-step reactions in microscopic environments, including biopolymer synthesis of oligonucleotides and peptides. The Heller method employs free field electrophoresis to transport analytes or reactants to selected micro-locations where they are effectively concentrated and reacted with the specific binding entities. Each micro-location of the Heller device has a derivatized surface for the covalent attachment of specific binding entities, which includes an attachment layer, a permeation layer, and an underlying direct current micro-electrode. The presence of the permeation layer prevents any electrochemically generated reagents from interacting with or binding to either the points of synthesis or to reagents that are electrophoretically transported to each synthesis site. Thus, all synthesis is due to reagents that are electrophoretically transported to each site of synthesis.

The Heller method, however, is severely limited by the use of electrophoretic transport. First, electrophoretic transport requires that the reactants be charged in order to be affected by the electric fields; however, conventional reactants of interest for combinatorial chemistry are usually uncharged molecules not useable in an electrophoretic system. Second, the Heller method does not, and cannot, address the large amount of chemical crosstalk that inherently occurs because of the spatial distribution of the electric fields involved in the electrophoretic transport of the reagents for binding. In a system utilizing electrophoresis, one cannot use protecting groups to protect the reactive functional groups at the microlocations since there is no mechanism for removing the protective groups; yet, the use of electrophoresis results in various binding entities and/or reactants being located throughout the solution used as they migrate, often coming into contact with unselected reaction sites. Thus, the combination of the lack of protecting groups and the spatial distribution of the electric fields inherent to electrophoresis allow such binding reactions to occur randomly, compromising the fidelity of any polymer being synthesized.

### SUMMARY OF THE INVENTION

In a first aspect, the present invention features methods for producing fine spotted arrays of molecules on an array. The methods comprise the following steps:

(a) immobilizing affinity anchors onto an array;

(b) preparing molecules of interest having affinity for the anchors;

(c) contacting the molecules of interest with the array; and

(d) washing the array to remove unbound molecules of interest.

In preferred embodiments, the affinity anchors are immobilized to the array electrochemically.

In a second aspect, the present invention features arrays of molecules comprising a plurality of affinity anchors and a plurality of molecules of interest bound thereto. In preferred embodiments, the array comprises at least about 100, and more preferably at least about 1000 electrodes.

### BRIEF DESCRIPTION OF THE FIGURES

FIG. 1 illustrates an embodiment of the methods of the present invention. A microarray of electrodes is coated with a porous membrane. Biotin is used as the molecular anchor. The microarray is immersed in a solution of biotin NHS-ester. The electrodes are biased as cathodes. The NHS-ester is activated by electrochemically generated reagents produced at the cathode, and the biotin is covalently attached to the membrane. The size of the biotin spot is dictated only by the size of the electrode. The amount of biotin immobilized is a function of how long the electrode is biased as a cathode. The chip is then immersed in streptavidin, which binds to the immobilized biotin with high affinity. DNA oligonucleotides may be synthesized with a terminal biotin. Individual oligonucleotides may be mechanically spotted onto a biotin/streptavidin anchor spot immobilized in the membrane, and the biotinylated oligonucleotides becomes chemically attached to the steptavidin. Once all of the oligonucleotides are spotted onto the array, the chip may be washed, removing extraneous DNA that may have diffused into areas outside of the streptavidin anchor.

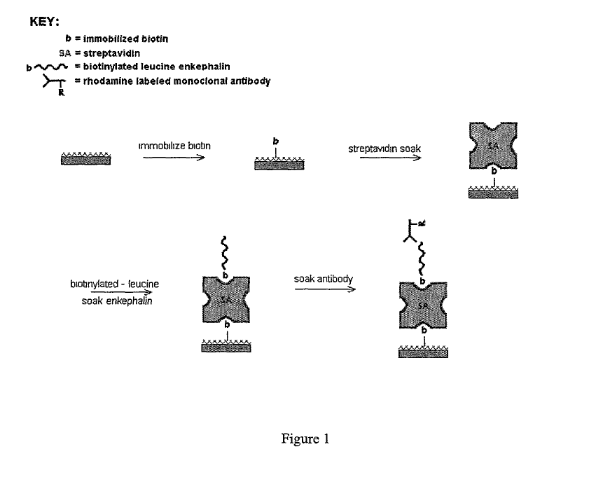

FIG. 2 represents results obtained from the experiment outlined in FIG. 1. Fluorescence depicted is the result of binding between the monoclonal antibody and leucine enkephalin.

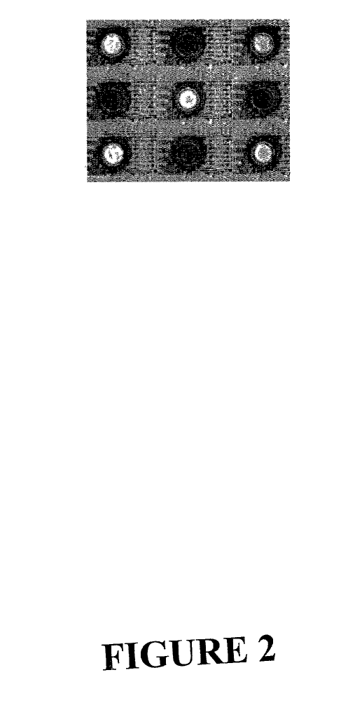

### DETAILED DESCRIPTION OF THE INVENTION

The present invention features methods for producing fine spotted arrays of molecules on an array. The methods comprise the following steps:

(a) immobilizing affinity anchors onto an array;

(b) preparing molecules of interest having affinity for the anchors;

(c) contacting the molecules of interest with the array; and

(d) washing the array to remove unbound molecules of interest.

In preferred embodiments, the affinity anchors are immobilized to the array electrochemically.

Preferably the affinity anchor is selected from the group consisting of a biotin, streptavidin, a peptide, an antibody and an oligonucleotide. Preferably, the molecule of interest is selected from the group consisting of an oligonucleotide, a peptide, an antibody and an antigen.

It is highly desirable to examine multiple molecular interactions simultaneously when studying complex biological systems. Such studies require large microarrays of many different biomolecular probes such as DNA oligonucleotides, peptides, proteins and antibodies. Increasing the density of probes in the array increases the number of interactions that can be examined simultaneously and reduces the amount of the biological sample required for the assay.

Arrays of biomolecular probes are commonly made by mechanically spotting each probe onto a membrane at a defined location. Unfortunately the porous nature of biocompatible membranes prevents all spotted probes from remaining in position on an array. Thus, closely spaced ultra-fine spots with well-defined edges and a precise amount of immobilized probe are difficult to prepare by conventional spotting techniques.

In a second aspect, the present invention features arrays of molecules comprising a plurality of affinity anchors and a plurality of molecules of interest bound thereto. In preferred embodiments, the array comprises at least about 100, and more preferably at least about 1000 electrodes.

Arrays

In preferred embodiments, the present invention features using an electrode array to electrochemically immobilize a well-defined array of affinity anchors onto a porous membrane, modify biomolecular probes so that the biomolecular probes bind to an anchor on the array, and sequentially spotting each biomolecular probe onto individual affinity anchor spots. After the probes of interest are spotted onto the array, the array is normally washed to remove probes outside the area of the immobilized anchor. The present method provide well-defined spots of biomolecular probes on an array. According to preferred embodiments of the methods of the present invention, the anchor molecule is attached to the array electrochemically so that the anchor molecule only becomes attached to the array over an active electrode. Hence, it is possible to more precisely control the amount of immobilized affinity anchor by adjusting the time the electrode is on. According to the methods of the present invention, the position of the affinity anchor spot is determined by the position of the electrode that immobilizes it. Moreover, the size of the affinity anchor spot is determined by the size of the electrode. Hence, the present invention allows arrays of biomolecular probes to be prepared with a spot size substantiality smaller than what is currently accessible through mechanical spotting.

The methods of the present invention are particularly applicable to the arrays described in U.S. Ser. Nos. 09/003,075 and 09/214,348, the disclosures of which are herein incorporated by reference. Such arrays are designed to allow synthesizing chemical compounds such as peptides at well-defined and individually addressable locations. Such arrays may be manufactured at low cost by contract fabricators using existing semiconductor manufacturing facilities. Such an array may be used to synthesize a pattern of molecules. First, the array may be coated with a biocompatible porous membrane that allows molecules to flow freely between a bulk solvent and an electrode. The array may then be immersed in a solution containing a precursor to an electrochemically-generated (ECG) reagent of interest. For peptide synthesis, this is preferably an ECG-reagent to remove amino protecting groups. A computer may then interface with the array to turn on the desired electrode pattern, and the precursor may be electrochemically converted into an active species. The electrochemically-generated (ECG) reagent, in turn, reacts with molecules immobilized to the membrane overlying the electrode.

A central feature of these preferred arrays is the ability to confine the ECG reagents to a region immediately adjacent to a selected microelectrode. The localization of ECG reagents may be achieved by exploiting the physical chemistry of the solution in which the microelectrode array is immersed. Such solutions usually contain buffers and scavengers that react with ECG reagents. However, the rate at which ECG reagents are produced can overwhelm the ability of the solution to react with them in the small local area immediately proximate to the microelectrode. As a result, chemistry that is mediated by ECG reagents occurs near selected microelectrodes, but there is no chemical crosstalk.

In some embodiments, the surface of these preferred arrays may be provided with a layer of linker molecules. Linker molecules allow for indirect attachment of monomers or pre-formed molecules to the substrate or a layer overlaying the substrate. The linker molecules are preferably attached to an overlaying layer via silicon-carbon bonds, using, for example, controlled porosity glass (CPG) as the layer material. Linker molecules also facilitate target recognition of the synthesized polymers. Furthermore, the linker molecules are preferably chosen based upon their hydrophilic/hydrophobic properties to improve presentation of synthesized polymers to certain receptors. For example, in the case of a hydrophilic receptor, hydrophilic linker molecules will be preferred so as to permit the receptor to approach more closely the synthesized polymer.

The linker molecules are preferably of sufficient length to permit polymers on a completed substrate to interact freely with binding entities exposed to the substrate. The linker molecules, when used, are preferably 10 to 1000 atoms long, and in especially preferred embodiments are about 650 atoms long to provide sufficient exposure of the functional groups to the binding entity. The linker molecules, which may be advantageously used in accordance with the invention include, for example, aryl acetylene, ethylene glycol oligomers containing from 2 to 10 monomer units, diamines, diacids, amino acids, and combinations thereof. Other linker molecules may be used in accordance with the different embodiments of the present invention and will be recognized by those skilled in the art in light of this disclosure.

The linker molecules are preferably of sufficient length to permit polymers such as peptides on a completed substrate to interact freely with binding entities exposed to the substrate. The linker molecules, when used, are most preferably about 650 atoms long to provide sufficient exposure of the functional groups to the binding entity. The linker molecules, which may be advantageously used in accordance with the invention include, for example, aryl acetylene, ethylene glycol oligomers containing from 2 to 10 monomer units, diamines, diacids, amino acids, and combinations thereof. Other linker molecules may be used in accordance with the different embodiments of the present invention and will be recognized by those skilled in the art in light of this disclosure.

According to another preferred embodiment, linker molecules may be provided with a cleavable group at an intermediate position, which group can be cleaved with an electrochemically generated reagent. This group is preferably cleaved with a reagent different from the reagent(s) used to remove the protective groups. This enables removal of the various synthesized polymers or peptides following completion of the synthesis by way of electrochemically generated reagents. In particular, derivatives of the acid labile 4,4′-dimethyoxytrityl molecules with an exocyclic active ester can be used in accordance with the present invention. These linker molecules can be obtained from Perseptive Biosystems, Framingham, Mass. More preferably, N-succinimidyl-4-[bis-(4-methoxyphenyl)-chloromethyl]-benzoate is used as a cleavable linker molecule during DNA synthesis. The synthesis and use of this molecule is described in A Versatile Acid-Labile Linker for Modification of Synthetic Biomolecules, by Brian D. Gildea, James M. Coull and Hubert Koester, Tetrahedron Letters, Volume 31, No. 49, pgs 7095-7098 (1990). Alternatively, other manners of cleaving can be used over the entire array at the same time, such as chemical reagents, light or heat.

The use of cleavable linker groups affords dissociation or separation of synthesized molecules, e.g., polymers or amino acid sequences, from the electrode array at any desired time. This dissociation allows transfer of the, for example, synthesized polymer or amino acid sequence, to another electrode array or to a second substrate. Obviously, those skilled in the art can contemplate several uses for transferring the molecules synthesized on the original electrode to a second substrate.

The preferred arrays used according to the present invention need not be in any specific shape, that is, the electrodes need not be in a square matrix shape. Contemplated electrode array geometries include: squares; rectangles; rectilinear and hexagonal grid arrays with any sort of polygon boundary; concentric circle grid geometries wherein the electrodes form concentric circles about a common center, and which may be bounded by an arbitrary polygon; and fractal grid array geometries having electrodes with the same or different diameters. Interlaced electrodes may also be used in accordance with the present invention. Preferably, however, the array of electrodes contains at least 100 electrodes in an at least 10×10 matrix. More preferably, the array of electrodes contains at least 400 electrodes in, for example, an at least 20×20 matrix. Even more preferably, the array contains at least 1024 or 2048 electrodes in, for example, an at least 64×32 matrix, and still more preferably, the array contains at least 204, 800 electrodes in, for example, an at least 640×320 array. Other sized arrays that may be used in accordance with the present invention will be readily apparent to those of skill in the art upon review of this disclosure.

Electrode arrays containing electrodes ranging in diameter from approximately less than 1 micron to approximately 100 microns (0.1 millimeters) are advantageously used in accordance with the present invention. Further, electrode arrays having a distance of approximately 10-1000 microns from center to center of the electrodes, regardless of the electrode diameter, are advantageously used in accordance with the present invention. More preferably, a distance of 50-100 microns exists between the centers of two neighboring electrodes.

The electrodes may be flush with the surface of the substrate. However, in accordance with a preferred embodiment of the present invention, the electrodes are hemisphere shaped, rather than flat disks. More specifically, the profile of the hemisphere shaped electrodes is represented by an arctangent function that looks like a hemisphere. Those skilled in the art will be familiar with electrodes of this shape. Hemisphere shaped electrodes help assure that the electric potential is constant across the radial profile of the electrode. That is, hemisphere shaped electrodes help assure that the electric potential is not larger near the edge of the electrode than in the middle of the electrode, thus assuring that the generation of electrochemical reagents occurs at the same rate at all parts of the electrode.

Electrodes that may be used in accordance with the invention may be composed of, but are not limited to, noble metals such as iridium and/or platinum, and other metals, such as, palladium, gold, silver, copper, mercury, nickel, zinc, titanium, tungsten, aluminum, as well as alloys of various metals, and other conducting materials, such as, carbon, including glassy carbon, reticulated vitreous carbon, basal plane graphite, edge plane graphite and graphite. Doped oxides such as indium tin oxide, and semiconductors such as silicon oxide and gallium arsenide are also contemplated. Additionally, the electrodes may be composed of conducting polymers, metal doped polymers, conducting ceramics and conducting clays. Among the noble metals, platinum and palladium are especially preferred because of the advantageous properties associated with their ability to absorb hydrogen, i.e., their ability to be “preloaded” with hydrogen before being used in the methods of the invention.

In accordance with other preferred embodiments of these arrays, one or more of the electrodes are proximate to a “getter” structure. Preferably the “getter” structure comprises a second electrode. The second electrode may be of any shape or size. However, it may function to scavenge electrochemically generated reagents alone or in conjunction with a scavenging solution and/or a buffering solution or it may function to reduce or eliminate diffusion of ions into nearby electric sources such as semiconductor circuitry. Such second electrodes may be made of the same material as the selected electrodes discussed above.

The electrode(s) used in accordance with the arrays may be connected to an electric source in any known manner. Preferred ways of connecting the electrodes to the electric source include CMOS switching circuitry, radio and microwave frequency addressable switches, light addressable switches, and direct connection from an electrode to a bond pad on the perimeter of a semiconductor chip. The placement of a “getter” structure in accordance with the description set forth above effectively prolongs the life of a semiconductor chip thereby making such a connection particularly advantageous.

CMOS switching circuitry involves the connection of each of the electrodes to a CMOS transistor switch. The switch is accessed by sending an electronic address signal down a common bus to SRAM (static random access memory) circuitry associated with each electrode. When the switch is “on”, the electrode is connected to an electric source. This is a preferred mode of operation.

Radio and microwave frequency addressable switches involve the electrodes being switched by a RF or microwave signal. This allows the switches to be thrown both with and/or without using switching logic. The switches can be tuned to receive a particular frequency or modulation frequency and switch without switching logic. Alternatively, the switches can use both methods.

Light addressable switches are switched by light. In this method, the electrodes can also be switched with and without switching logic. The light signal can be spatially localized to afford switching without switching logic. This is accomplished, for example, by scanning a laser beam over the electrode array; the electrode being switched each time the laser illuminates it. Alternatively, the whole array can be flood illuminated and the light signal can be temporally modulated to generate a coded signal. However, switching logic is required for flood illumination.

One can also perform a type of light addressable switching in an indirect way. In this method, the electrodes are formed from semiconductor materials. The semiconductor electrodes are then biased below their threshold voltage. At sufficiently low biases, there is no electrochemistry occurring because the electrons do not have enough energy to overcome the band gap. The electrodes that are “on” will already have been switched on by another method. When the electrodes are illuminated, the electrons will acquire enough energy from the light to overcome the band gap and cause electrochemistry to occur.

Thus, an array of electrodes can be poised to perform electrochemistry whenever they are illuminated. With this method, the whole array can be flood illuminated or each electrode can be illuminated separately. This technique is useful for very rapid pulsing of the electrochemistry without the need for fast switching electronics. Direct connection from an electrode to a bond pad on the perimeter of the semiconductor chip is another possibility, although this method of connection could limit the density of the array.

Electrochemical generation of the desired type of chemical species requires that the electric potential of each electrode have a certain minimum value. That is to say, a certain minimum potential is necessary, which may be achieved by specifying either the voltage or the current. Thus, there are two ways to achieve the necessary minimum potential at each electrode: either the voltage may be specified at the necessary value or the current can be determined such that it is sufficient to accommodate the necessary voltage. The necessary minimum potential value will be determined by the type of chemical reagent chosen to be generated. One skilled in the art can easily determine the necessary voltage and/or current to be used based on the chemical species desired. The maximum value of potential that can be used is also determined by the chemical species desired. If the maximum value of potential associated with the desired chemical species is exceeded, undesired chemical species may be resultantly produced.

Synthesis Methods

The present invention, in preferred embodiments, features electrochemically immobilizing an affinity anchor to an array. Exemplary affinity anchors within the scope of the present invention include biotin, streptavidin, oligonucleotides, peptides, antibodies, modified peptides having oligonucleotide ligands attached thereto, and the like. Those of skill in the art readily understand that any molecule having suitable binding affinity to the molecule to be localized on the array may be used.

When the affinity anchor is a peptide, antigen, antibody or oligonucleotide, the affinity anchor molecule may be immobilized on the array using the methods set forth in U.S. Ser. Nos. 09/003,075 and 09/214,348, the disclosures of which are herein incorporated by reference. An array according to the present invention may be used to synthesize a pattern of molecules. First, the array is coated with a biocompatible porous membrane that allows molecules to flow freely between the bulk solvent and the electrode. The array is then immersed in a solution containing an inactive precursor to the electrochemically generated (ECG) reagent of interest. For peptide synthesis, this would be an ECG-reagent to remove amino protecting groups. A computer then turns on the desired electrode pattern, and the precursor is electrochemically converted into the active species. The ECG-reagent, in turn, reacts with molecules immobilized to the membrane overlying the electrode. By buffering the solution properly, diffusion of the ECG-reagent beyond the area of the active electrode is eliminated.

The method of the present invention preferably utilizes a method for electrochemical placement of a material at a specific location on a substrate as described in U.S. patent application Ser. Nos. 09/003,075 and 09/214,348, the disclosures of which are herein incorporated by reference, and in international patent application numbers PCT/US97/11463 and PCT/US99/00599, comprising the steps of: providing a substrate having at its surface at least one electrode that is proximate to at least one molecule that is reactive with an electrochemically generated reagent, applying a potential to the electrode sufficient to generate electrochemical reagents capable of reacting to the at least one molecule proximate to the electrode, and producing a chemical reaction thereby. Such method allows production of an array of affinity anchors such as oligonucleotides and peptides.

In other preferred embodiments, the present invention utilizes a method for electrochemical synthesis of an array of separately formed oligonucleotides or peptides on a substrate, which comprises the steps of: placing a buffering or scavenging solution in contact with an array of electrodes that is proximate to a substrate surface, said surface being proximate to one or more molecules bearing at least one protected chemical functional group attached thereto, selectively deprotecting at least one protected chemical functional group on at least one of the molecules; bonding a first monomer, normally a nucleotide or amino acid, having at least one protected chemical functional group to one or more deprotected chemical functional groups of the molecule; selectively deprotecting a chemical functional group on the bonded molecule or another of the molecules bearing at least one protected chemical functional group; bonding a second monomer, normally a nucleotide or amino acid, having at least one protected chemical functional group to a deprotected chemical functional group of the bonded molecule or the other deprotected molecule; and repeating the selective deprotection of a chemical functional group on a bonded protected monomer or a bonded protected molecule and the subsequent bonding of an additional monomer, normally a nucleotide or amino acid, to the deprotected chemical functional group until at least two separate polymers, normally oligonucleotides or peptides, of desired length are formed on the substrate surface.

In additional preferred embodiments, the present invention utilizes a method for electrochemical synthesis of an array of separately formed polymers on a substrate, which comprises the steps of: placing a buffering or scavenging solution in contact with an array of electrodes that is proximate to a substrate surface, said surface being proximate to one or more molecules bearing at least one protected chemical functional group attached thereto, selectively deprotecting at least one protected chemical functional group on at least one of the molecules; bonding a first monomer having at least one protected chemical functional group to one or more deprotected chemical functional groups of the molecule; selectively deprotecting a chemical functional group on the bonded molecule or another of the molecules bearing at least one protected chemical functional group; bonding a second monomer having at least one protected chemical functional group to a deprotected chemical functional group of the bonded molecule or the other deprotected molecule; and repeating the selective deprotection of a chemical functional group on a bonded protected monomer or a bonded protected molecule and the subsequent bonding of an additional monomer to the deprotected chemical functional group until at least two separate polymers of desired length are formed on the substrate surface. According to the present invention, the monomer or molecule is normally a nucleotide or amino acid and the polymer is an oligonucleotide or peptide.

By using these electrochemical techniques, it is possible to place monomers, both those that can be used for polymer synthesis and those that can be decorated, and pre-formed molecules at small and precisely known locations on a substrate. It is therefore possible to synthesize peptides of a known amino acid sequence or oligonucleotides of a known nucleic acid sequence at selected locations on a substrate. For example, in accordance with the presently disclosed invention, one can place amino acids at selected locations on a substrate to synthesize desired sequences of amino acids in the form of peptides. Also, in accordance with the presently disclosed invention, one can localize an affinity anchor such as biotin or streptavidin at a discrete location on an array.

Preferred embodiments of the methods of synthesis described herein use a buffering or scavenging solution in contact with each electrode, which is buffered towards the electrochemically generated reagents, in particular, towards protons and/or hydroxyl ions, and that actively prevents chemical cross-talk caused by diffusion of the electrochemically generated ions from one electrode to another electrode in an array. For example, when an electrode exposed to an aqueous or partially aqueous media is biased to a sufficiently positive (or negative) potential, protons (or hydroxyl ions) are produced as products of water hydrolysis. Protons, for example, are useful for removing electrochemical protecting groups from several molecules useful in combinatorial synthesis, for example, peptides.

In order to produce separate and pure peptides, it is desirable to keep these protons (or hydroxyl ions) confined to the area immediately proximate the selected electrode(s) in order to minimize, and, if possible to eliminate, chemical cross-talk between nearby electrodes in an array. The spatial extent of excursion of electrochemically generated reagents can be actively controlled by the use of a buffering or scavenging solution that reacts with the reagents that move away from the selected electrodes, thus preventing these reagents from reacting at a nearby electrode.

Another technique for confining these electrochemically generated reagents to the area immediately proximate the selected electrode(s) is to place a “getter” structure in proximity to the selected electrode(s) and substantially exposed to the external environment. Such a “getter” structure may be used in conjunction with or in place of a scavenging solution. A “getter” structure may be designed of any suitable material and formed into any suitable shape or size as skilled artisans will readily appreciate. The most important criteria for such a “getter” structure is that it function to scavenge electrochemically generated reagents that may diffuse away from the selected electrode(s). The “getter” structure may function passively by reacting chemically with the electrochemically generated reagents. Alternatively, the “getter” structure may function actively to scavenge the electrochemically generated reagents. This may be performed by applying sufficient potential to the “getter” structure to cause electrochemical scavenging. Another function of the “getter” structure may be to prevent the diffusion of ions toward or into circuitry such as transistors that may be operably linked to the selected electrode(s). In accordance with this function, the “getter” structure may be placed substantially at the interface between an insulating dielectric and a metallization layer operably linked to the selected electrode(s).

In embodiments wherein the affinity anchor is a peptide or oligonucleotide, the substrate in the invention is proximate to at least one electrode, i.e., an electrically conducting region of the substrate that is substantially surrounded by an electrically insulating region. The electrode(s), by being “proximate” to the substrate, can be located at the substrate, i.e., embedded in or on the substrate, can be next to, below, or above the substrate, but need to be in close enough proximity to the substrate so that the reagents electrochemically generated at the electrode(s) can accomplish the desired deprotection of the chemical functional groups on the monomer(s) and/or molecule(s).

In addition to being proximate to at least one electrode, the substrate has on a surface thereof, at least one molecule, and preferably several molecules, bearing at least one chemical functional group protected by an electrochemically removable protecting group. These molecules bearing protected chemical functional groups also need to be proximate to the electrode(s). In this regard, the molecules on the surface of the substrate need to be in close enough proximity to the electrode(s) so that the electrochemical reagents generated at the electrode can remove the protecting group from at least one protected functional group on the proximate molecule(s).

The molecules bearing a protected chemical functional group that are attached to the surface of the substrate may be selected generally from monomers, linker molecules and pre-formed molecules. Preferably, the molecules attached to the surface of the substrate include monomers, nucleotides, amino acids, peptides, and linker molecules. All of these molecules generally bond to the substrate by covalent bonds or ionic interactions. Alternatively, all of these molecules can be bonded, also by covalent bonds or ionic interactions, to a layer overlaying the substrate, for example, a permeable membrane layer, which layer can be adhered to the substrate surface in several different ways, including covalent bonding, ionic interactions, dispersive interactions and hydrophilic or hydrophobic interactions. In still another manner of attachment, a monomer or pre-formed molecule may be bonded to a linker molecule that is bonded to either the substrate or a layer overlaying the substrate.

The monomers, linker molecules and pre-formed molecules used herein, preferably amino acids or nucleotides, are preferably provided with a chemical functional group that is protected by a protecting group removable by electrochemically generated reagents. If a chemical functional group capable of being deprotected by an electrochemically generated reagent is not present on the molecule on the substrate surface, bonding of subsequent monomers or pre-formed molecules cannot occur at this molecule. Preferably, the protecting group is on the distal or terminal end of the linker molecule, monomer, or pre-formed molecule, opposite the substrate. That is, the linker molecule preferably terminates in a chemical functional group, such as an amino or carboxy acid group, bearing an electrochemically removable protective group. Chemical functional groups that are found on the monomers, linker molecules and pre-formed molecules include any chemically reactive functionality. Usually, chemical functional groups are associated with corresponding protective groups and will be chosen or utilized based on the product being synthesized. The molecules of the invention bond to deprotected chemical functional groups by covalent bonds or ionic interactions.

Monomers, particularly oligonucleotides and amino acids, used in accordance with the methods of the present invention to synthesize the various polymers, particularly oligonucleotides and peptides, contemplated for use as affinity anchors include all members of the set of small molecules that can be joined together to form a polymer. This set includes, but is not limited to, the set of common L-amino acids, the set of D-amino acids and the set of synthetic amino acids. Monomers include any member of a basis set for synthesis of a polymer. For example, trimers of L-amino acids form a basis set of approximately 8000 monomers for synthesis of polypeptides. Different basis sets of monomers may be used at successive steps in the synthesis of a polymer. The number of monomers that can be used in accordance with the synthesis methods can vary widely, for example from 2 to several thousand monomers can be used, but in more preferred embodiments, the number of monomers will range from approximately 4 to approximately 200, and, more preferably, the number of monomers will range from 4-20.

Furthermore, essentially any pre-formed molecule can be bonded to the substrate, a layer overlaying the substrate, a monomer or a linker molecule and serve as an affinity anchor. Pre-formed molecules include, for example, proteins, including in particular, receptors, enzymes, ion channels, and antibodies, nucleic acids, antigens and the like. Pre-formed molecules are, in general, formed at a site other than on the substrate. In a preferred embodiment, a pre-formed molecule is bonded to a deprotected functional group on a molecule, monomer, or another pre-formed molecule. In this regard, a pre-formed molecule that is already attached to the substrate may additionally bear at least one protected chemical functional group to which a monomer or other pre-formed molecule may bond, following deprotection of the chemical functional group.

Protective groups are materials that bind to a monomer, a linker molecule or a pre-formed molecule to protect a reactive functionality on the monomer, linker molecule or pre-formed molecule, which may be removed upon selective exposure to an activator, such as an electrochemically generated reagent. Protective groups that may be used in accordance with the present invention preferably include all acid and base labile protecting groups. For example, peptide amine groups are preferably protected by t-butyloxycarbonyl (BOC) or benzyloxycarbonyl (CBZ), both of which are acid labile, or by 9-fluorenylmethoxycarbonyl (FMOC), which is base labile. Additionally, hydroxy groups on phosphoramidites may be protected by dimethoxytrityl (DMT), which is acid labile. Exocyclic amine groups on nucleosides, in particular on phosphoramidites, are preferably protected by dimethylformamidine on the adenosine and guanosine bases, and isobutyryl on the cytidine bases, both of which are base labile protecting groups. This protection strategy is known as fast oligonucleotide deprotection (FOD). Phosphoramidites protected in this manner are known as FOD phosphoramidites.

Additional protecting groups that may be used in accordance with the present invention include acid labile groups for protecting amino moieties: tert-butyloxycarbonyl, tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2)oxycarbonyl, α,α-dimethyl-3,5-dimethyloxybenzyloxy-carbonyl, 2-phenylpropyl(2)oxycarbonyl, 4-methyloxybenzyloxycarbonyl, benzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups for protecting amino moieties: 9-fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio)carbonyl; as groups for protecting amino moieties that are labile to miscellaneous reagents, the appropriate agent is listed in parenthesis after the group: phthaloyl (hydrazine), trifluoroacetyl (piperidine), and chloroacetyl (2-aminothiophenol); acid labile groups for protecting carboxylic acids: tert-butyl ester; acid labile groups for protecting hydroxyl groups: dimethyltrityl; and basic labile groups for protecting phosphotriester groups: cyanoethyl.

The molecules of the invention, i.e., the monomers, linker molecules and pre-formed molecules, can be attached directly to the substrate or can be attached to a layer or membrane of separating material that overlays the substrate. That is, the affinity anchors of the present invention may be attached to a layer or membrane of separating material that overlays the substrate. Materials that can form a layer or membrane overlaying the substrate, such that molecules can be bound there for modification by electrochemically generated reagents, include: controlled porosity glass (CPG); generic polymers, such as, teflons, nylons, polycarbonates, polystyrenes, polyacylates, polycyanoacrylates, polyvinyl alcohols, polyamides, polyimides, polysiloxanes, polysilicones, polynitriles, polyelectrolytes, hydrogels, epoxy polymers' melamines, urethanes and copolymers and mixtures of these and other polymers; biologically derived polymers, such as, polysaccharides, polyhyaluric acids, celluloses, and chitons; ceramics, such as, alumina, metal oxides, clays, and zeolites; surfactants; thiols; self-assembled monolayers; porous carbon; and fullerine materials. The membrane can be coated onto the substrate by spin coating, dip coating or manual application, or any other art acceptable form of coating.

Reagents that can be generated electrochemically at the electrodes fall into two broad classes: oxidants and reductants. There are also miscellaneous reagents that are useful in accordance with the invention. Oxidants that can be generated electrochemically include iodine, iodate, periodic acid, hydrogen peroxide, hypochlorite, metavanadate, bromate, dichromate, cerium (IV), and permanganate. Reductants that can be generated electrochemicaily include chromium (II), ferrocyanide, thiols, thiosulfate, titanium (II), arsenic (III) and iron (II). The miscellaneous reagents include bromine, chloride, protons and hydroxyl ions. Among the foregoing reagents, protons, hydroxyl ions, iodine, bromine, chlorine and the thiols are preferred.

In accordance with preferred embodiments of the methods of synthesizing affinity anchors of the present invention, a buffering and/or scavenging solution is in contact with each electrode. The buffering and/or scavenging solutions that may be used in accordance with the invention are preferably buffered toward, or scavenge, ions such as protons and/or hydroxyl ions, although other electrochemically generated reagents capable of being buffered and/or scavenged are clearly contemplated. The buffering solution functions to prevent chemical cross-talk due to diffusion of electrochemically generated reagents from one electrode in an array to another electrode in the array, while a scavenging solution functions to seek out and neutralize/deactivate the electrochemically generated reagents by binding or reacting with them. Thus, the spatial extent of excursion of electrochemically generated reagents can be actively controlled by the use of a buffering solution and/or a scavenging solution. In accordance with the invention, the buffering and scavenging solutions may be used independently or together. Preferably, a buffering solution is used because the capacity of a buffering solution is more easily maintained, as compared with a scavenging solution.

Buffering solutions that can be used in accordance with the present invention include all electrolyte salts used in aqueous or partially aqueous preparations. Buffering solutions preferably used in accordance with the present invention include: acetate buffers, which typically buffer around pH 5; borate buffers, which typically buffer around pH 8; carbonate buffers, which typically buffer around pH 9; citrate buffers, which typically buffer around pH 6; glycine buffers, which typically buffer around pH 3; HEPES buffers, which typically buffer around pH 7; MOPS buffers, which typically buffer around pH 7; phosphate buffers, which typically buffer around pH 7; TRIS buffers, which typically buffer around pH 8; and 0.1 M KO in solution, which buffers the iodine concentration by the equilibrium reaction I₂+I⁻=I₃⁻, the equilibrium coefficient for this reaction being around 10⁻².

Alternatively, or in combination with a buffering solution, a scavenging solution may be used that contains species such as ternary amines that function as proton scavengers or sulfonic acids that function as hydroxyl ion scavengers in nonaqueous media. The rate at which a reagent/species is scavenged depends both on the intrinsic rate of the reaction occurring and on the concentration of the scavenger. For example, solvents make good scavengers because they are frequently present in high concentrations. Most molecules scavenge in a nonselective way, however, some molecules, such as superoxide dismutase and horseradish peroxidase, scavenge in a selective manner.

Of particular interest to the present invention are scavenger molecules that can scavenge the different reactive species commonly generated, for example, by water hydrolysis at electrodes, including hydroxyl radicals, superoxides, oxygen radicals, and hydrogen peroxide. Hydroxyl radicals are among the most reactive molecules known, their rate of reaction is diffusion controlled, that is, they react with the first reactant/species they encounter. When hydroxyl radicals are generated by water hydrolysis, the first molecule they usually encounter is a water molecule. For this reason, water is a rapid and effective scavenger of hydroxyl radicals. Superoxides are also a relatively reactive species, but can be stable in some nonaqueous or partially aqueous solvents. In aqueous media, superoxides rapidly react with most molecules, including water. In many solvents, they can be scavenged selectively with superoxidase dismutase.

Oxygen radicals are a family of oxygen species that exist as free radicals. They can be scavenged by a wide variety of molecules such as water or ascorbic acid. Hydrogen peroxide is a relatively mild reactive species that is useful, in particular, in combinatorial synthesis. Hydrogen peroxide is scavenged by water and many types of oxidizing and reducing agents. The rate at which hydrogen peroxide is scavenged depends on the redox potential of the scavenger molecules being used. Hydrogen peroxide can also be scavenged selectively by horseradish peroxidase. Another electrochemically generated species that can be scavenged is iodine. Iodine is a mild oxidizing reagent that is also useful for combinatorial synthesis. Iodine can be scavenged by reaction with hydroxyl ions to form iodide ions and hypoiodite. The rate at which iodine is scavenged is pH dependent; higher pH solutions scavenge iodine faster. All of the scavenger molecules discussed above may be used in accordance with the present invention. Other scavenger molecules will be readily apparent to those skilled in the art upon review of this disclosure.

In accordance with the methods of synthesizing affinity anchors of the present invention, the buffering solutions are preferably used in a concentration of at least 0.01 mM. More preferably, the buffering solution is present in a concentration ranging from 1 to 100 mM, and still more preferably, the buffering solution is present in a concentration ranging from 10 to 100 mM. Most preferably, the buffering solution concentration is approximately 30 mM. A buffering solution concentration of approximately 0.1 molar, will allow protons or hydroxyl ions to move approximately 100 angstroms before buffering the pH to the bulk values. Lower buffering solution concentrations, such as 0.00001 molar, will allow ion excursion of approximately several microns, which still may be acceptable distance depending on the distance between electrodes in an array.

In accordance with the methods of the present invention, the concentration of scavenger molecules in a solution will depend on the specific scavenger molecules used since different scavenging molecules react at different rates. The more reactive the scavenger, the lower the concentration of scavenging solution needed, and vice versa. Those skilled in the art will be able to determine the appropriate concentration of scavenging solution depending upon the specific scavenger selected.

The at least one electrode proximate the substrate of the invention is preferably an array of electrodes. Arrays of electrodes of any dimension may be used, including arrays containing up to several million electrodes. Preferably, multiple electrodes in an array are simultaneously addressable and controllable by an electrical source. More preferably, each electrode is individually addressable and controllable by its own electrical source, thereby affording selective application of different potentials to select electrodes in the array. In this regard, the electrodes can be described as “switchable”. Binding Molecules of Interest Exemplary affinity anchors within the scope of the present invention include biotin, streptavidin, oligonucleotides, peptides, antibodies, modified peptides having oligonucleotide ligands attached thereto, and the like. Those of skill in the art readily understand that any molecule having suitable binding affinity to the molecule to be localized on the array may be used. Once the affinity anchors of the present invention are localized to the array, preferably by electrochemical immobilization, the array may be contacted with the molecules of interest to be localized thereto.

Where the affinity anchor is a peptide, the molecule of interest binds by ligand interaction. Where the affinity anchor is an antibody, the molecule of interest is a corresponding antigen or is labeled with a corresponding antigen. Where the affinity anchor is an oligonucleotide, the molecule of interest binds by hybridization interaction. Where the affinity anchor is biotin, the molecule of interest is labeled with streptavidin and vice versa.

The molecules of interest may be contacted with the affinity anchors under suitable binding or hybridizing conditions as are well known to those skilled in the art. Once the molecules of interest have bound to the affinity anchors, the excess, unbound molecules of interest may be removed by any suitable washing step known to those of skill in the art.

### DESCRIPTION OF THE PREFERRED EMBODIMENTS

The following are provided purely by way of example and are not intended to limit the scope of the present invention.

### EXAMPLE 1

### Preparation of Binding Array

A chip was immersed in a solution consisting of biotin-N-hydroxysuccinimide ester (3.5 mg) in DMF (200 μL) and 800 μL of a solution comprising the following dissolved in methanol: vitamin B₁₂ (2.8 mg/mL) and tetramethylamonium nitrate (TBAN, 13.3 mg/mL). An electrical potential of 2.4 V was applied to the desired pattern of electrodes for 5 minutes. The chip was then rinsed with ethanol, followed by water, and finally by ethanol. The chip was then immersed in a solution of streptavidin (50 μg/mL in 10 mM phostphate buffer, pH 7) for 15 minutes and rinsed. The chip was then exposed to a solution of biotinylated peptide of the sequence YGGFL dissolved in DMF and diluted with 9 equivalents of phosphate buffer (10 mM, pH 7). After 1 hour, the chip was rinsed, and exposed to a solution of anti-βendorphin (clone 3-E7, Boeringer Mannheim, approximately 250 nM in 10 mM phospahte buffer, pH 7) that had been derivatized with the fluorescent dye rhodamine. The chip was given a final rinse, and imaged under an epifluorescent microscope.

Although the invention has been described with reference to the presently preferred embodiments, it should be understood that various modifications can be made without departing from the spirit of the invention. Accordingly, the invention is limited only by the following claims.

What is claimed is:

1. A method for producing fine spotted arrays of molecules comprising the following steps:
(a) immobilizing a plurality of affinity anchors onto the array;
(b) preparing a plurality of molecules of interest having affinity for the anchors;
(c) contacting the molecules of interest with the array; and
(d) washing the array to remove unbound molecules of interest.

2. The method of claim 1 wherein the affinity anchors are selected from the group consisting of biotin, streptavidin, a peptide, an antibody and an oligonucleotide.

3. The method of claim 1 wherein the molecules of interest are selected from the group consisting of a peptide, an antibody and an oligonucleotide.

4. The method of claim 1 wherein the affinity anchor molecules are attached to the array by a linker molecule.

5. The method of claim 1 wherein the affinity anchor molecules are attached to the array by a cleavable linker molecule.

6. The method of claim 1 wherein the array comprises at least about 100 electrodes.

7. The method of claim 1 wherein the array comprises at least about 1000 electrodes.

8. An array of molecules produced according to the method of claim 1.

9. An array of molecules comprising a plurality of affinity anchors and a plurality of molecules of interest bound to the affinity anchors.

10. An array according to claim 9 wherein the plurality of affinity anchors are selected from the group consisting of a biotin, streptavidin, a peptide, an antibody and an oligonucleotide.

11. An array according to claim 9 wherein the plurality of molecules of interest are selected from the group consisting of a peptide, an antibody and an oligonucleotide.

12. An array according to claim 9 further comprising at least about 1000 electrodes.
